(19) United States
(12) Patent Application Publication
(10) Pub. No.: US 2022/0202550 A1
(43) Pub. Date: Jun. 30, 2022

(54) DEVICE FOR THE EXTERNAL SUPPORT OF THE FEMALE URETHRA UNDER STRESS

(71) Applicant: EURL CORNIER, Paris (FR)

(72) Inventor: Edgard Cornier, Paris (FR)

(21) Appl. No.: 17/574,886

(22) Filed: Jan. 13, 2022

Related U.S. Application Data
(63) Continuation-in-part of application No. 17/143,295, filed on Jan. 7, 2021.

(30) Foreign Application Priority Data
Jan. 17, 2020 (FR) 2000430
Nov. 17, 2020 (FR) 2011794
Jun. 23, 2021 (FR) 2106711

(51) Int. Cl. A61F 2/00; A41B 9/04; A41B 9/14

(52) U.S. Cl. CPC A61F 2/0009; A41B 9/04; A41B 2400/32; A41B 2300/30; A41B 9/14

(57) ABSTRACT

Device for external directional support of the female urethra under stress, consisting of a rigid cushion (1) fixed to a rigid counter-support (2) placed in the rear part of a belt or briefs, and comprising a partial rear perineal support part, the rigid counter-support (2) being connected at the front by adjustable straps (2) supporting it to a belt (5) or briefs whose rear periphery is rigidified by an insert or other non-folding material.

### FIELD OF THE INVENTION

The present invention refers to a device for externally maintaining the female urethra under stress.

According to a new postulate (http://perineologie-vectorielle.com) developed by the inventor, the abdominal forces are not directed vertically to the pelvic floor. They are reflected from the bone surface and muscle structure of the small pelvis. These forces are physically redirected forward. Friction between tissues bonded together is retained by the young connective tissue. (reference A2499EC European Journal of Obstetrics & Gynecology and Reproductive Biology 234 (2019)—From tissue viscoelasticity to vectorial perineology—Edgard Cornier 1, 2, * et al.) the pressures produce quasi-static plicature movements between the organs. The plicature of the organs creates areas called “functional” areas. These areas obstruct leakage. The invention consists in correcting these force returns to recreate the functional plicature of the organs of the small pelvis. A posterior support used with the redirection of these force vectors, will allow the flexion of the female urethra, its closure, and the healing of urinary stress incontinence. The remainder of the pelvis, both centrally and laterally (the labia majora and adjacent tissues) should not be retained and should remain free to move.

### STATE OF THE TECHNIQUE

Approximately 20% of young women and 77% at the age of tissue sagging suffer from urinary incontinence disorders (Urinary Incontinence in Women: A Review. Lukacz E S, Santiago-Lastra Y, Albo M E, Brubaker L. JAMA. 2017; 3; 24(318): 10-1604.)

The available therapeutic tools are few and most affected women are obliged to resort to wearing absorbent diapers.

Muscle re-education alone is not very effective in the long term and can be restrictive. It needs supplementing by different methods, calling for a personal investment that is difficult to handle in the long term. Stopping training is accompanied by recurrence.

Kegel Exercises, Biofeedback, Electrostimulation, and Peripheral Neuro-modulation Improve Clinical Symptoms of Fecal Incontinence and Affect Specific Physiological Targets: A Randomized Controlled Trial. Mundet L, Rofes L, Ortega O, Cabib C, Clavé P. J Neurogastroenterol Motil. 2020 October.

The use of intra-vaginal pessaries is uncomfortable, poorly monitored, and not very effective on bladder weakness. Pessary use in stress urinary incontinence: a review of advantages, complications, patient satisfaction, and quality of life. Al-Shaikh G, Syed S, Osman S, Bogis A, Al-Badr A. Int J Womens Health. 2018

Surgery is often used to insert a direct suburethral support sling to cause forced flexion of the urethra, to close it under stress. This surgery has been widely used by Urologists. But the high percentage of complications has now outlawed or very strongly restricted its use. (Survey on surgery for stress urinary incontinence in an era mid-urethral slings are being questioned. D'hulster A S, Housmans S, Spaans W, Van der Aa F, Slabbaert K. Milani A L, Deprest J. Int Urogynecol J. 2020 April; 31(4):695-702).

The following documents cover the support of the whole perineum according to the technological background: EP 2 158 882, US 2015/034074, JP 2014 1510-20. These earlier patents propose devices placed over the entire perineum and held in place by straps. They are designed to prevent prolapse displacement by pushing it in general, using a convex or concave device, towards the inside of the whole perineum.

These devices tend to be wide, bearing on the outside of the vulva, on the labia majora. They sometimes have a gutter in front to provide an outlet for urine. None of them claims to be a treatment for bladder weakness in women.

These devices lift all the organs of the small pelvis far beyond the posterior area of the perineum and lift all the pelvic tissues. They are designed to control large organ prolapses. They have no plicature effect. As a result, they may even aggravate bladder weakness once again. (Reference: Commentary on ‘De novo urinary incontinence after pelvic organ prolapse surgery-a national database study’. Abdelrahman A. Int Urogynecol J. 2020 February; 31(2):309)

In a more detailed manner, the postulate or the usual hypothesis refers to the anatomy documents (Kamina Anatomie Clinique Tome 4-2^{nd} Ed.—4^{th} print run-Maloine 2012-383p). According to these documents, the viscera are solidly supported and suspended (p. 250).

FIG. 7 shows an approach that uses the postulate of the existence of a pelvic support floor. The known devices that are convex, cover the entire perineum to support the pressure forces that are directly directed towards the perineum.

According to the state of the art stated above, the known devices are convex, flexible, wide and long and cover the entire perineal surface (FIGS. 7 and 8).

In the case of the U.S. Pat. No. 8,677,910 document, the male device is in the form of a projection with a flat top, comprehensive, rigid, wide and long covering the entire perineal surface.

### SUMMARY OF THE INVENTION

The present invention aims at developing a directional device that forces the urethra to flex. This allows the plication of the urethra during an abdominal effort because of a partial and posterior support.

Accordingly, the invention refers to a device for externally maintaining the female urethra under stress, characterized in that it comprises
a rigid rectangular cushion, consisting of a short narrow and rigid panel that maintains its flat form, with a back strap and two front straps

and covering the posterior part of the perineum.
an abdominal belt with one rear and two front attachment points which adjustably receive respectively the one rear and two front straps to hold the rigid cushion against the rear and lower perineal area, starting from the coccyx.

The directional support device according to the invention is able to redirect the abdominal pressure forces during abdominal effort thanks to the rear placement of this rigid, flat and narrow counter-support, constituted by the rigid and short panel maintaining its flat form, placed in front of the coccyx, not covering the entire perineal surface area and remaining at some distance from the urinary orifice. It allows the occlusive plication of the functional area of the urethra, or for vesico urethral angulation and closure. This plication suffices to prevent urinary leakages.

This innovative concept has not been accepted without tests that proved its effectiveness. Thus, a scientific personality could evaluate its innovative value.

Thanks to the maintenance device as per the invention, the pressure produces quasi-static movements of plication of the organs between them. (reference A2499EC European Journal of Obstetrics & Gynecology and Reproductive Biology 234 (2019)—From tissue viscoelasticity to verctorial perineology—Edgar Cornier 1, 2, *, et al.). The Plication of the organs creates ‘functional’ areas. It is these areas that prevent leakages. The invention consists of correcting this return of forces in order to recreate the functional plication of the organs of the small pelvis. A posterior support will enable the redirection of these force vectors, allow the flexion of the female urethra, its closure, and the healing of urinary stress incontinence. The rest of the pelvis, at the centre as well as laterally (the labia majora and adjacent tissues) should not be retained and should remain free to move.

To summarise, the device according to the invention enables the return of forces that causes the plication and the stacking of tissues which leads to a physiological occlusion of the urethra preventing urinary leakage.

In one embodiment, the belt has a rear reinforcement supporting the rear attachment point.

In another embodiment, the belt is combined with briefs and in particular a cushion with a short narrow and rigid panel that maintains its flat form is integrated in the intermediate part of the briefs connecting the front and back parts.

The briefs have a reinforced rear part at the waistband supporting the rear attachment point and a rigid rear part connected to the reinforcement of the gluteal cleft part of the briefs.

Another advantageous characteristic is that the briefs have a double wall at the front, forming a passage route for each of the front straps to reach the two adjustable attachment points on the front sides.

Advantageously, the front adjustable attachment points each consist of a double ring over the exit opening of the corresponding strap. This embodiment allows easy and simple adjustable fastening.

Advantageously, the rear adjustable attachment point includes a through buckle attached to the rear reinforcement and the strap has a toothed snap end for hooking the strap into one of the teeth of the buckle to complete a fixed adjustment.

Once secured, this adjustment is maintained while the adjustment at the front attachment points continues to be possible, allowing tightening or loosening at any time depending on the need or to cater to comfort.

The device according to the invention is adjustable thanks to the adjustment of the front tension belt. Depending on the intensity of the abdominal forces, the return of the pressure forces can be more or less redirected upwards towards the pubic bony structure. The device blocked at the back by the bony support of the coccyx can thus be adjusted and adapted throughout the day. The adjustable tension of the front straps allows it. In fact, the device is narrow and thus mobile between the other lateral bony reliefs of the branches of the pubis and the ischium.

Particularly advantageously, the cushion, in the form of a rigid and short panel maintaining its flat form, is integrated in the rear lower region of the intermediate part of the briefs, while the front of the cushion has the two straps passing through the two routing channels of the double front wall to join the two front attachment points.

This embodiment is particularly advantageous because it is practical and comfortable.

Its length does not exceed the two-thirds of the length of the perineum. It leaves the front one-third of the perineum free.

This device is held in place by different possible means like briefs or an abdominal belt.

The average dimensions of the cushion, in the form of a rigid and short panel maintaining its flat form, are of the order of: length from 5 cm to 11 cm and width of 2 cm.

A flexible envelope comprising a biocompatible protection surrounds the rigid part, consisting of the rigid and short panel maintaining its flat form.

The rigid panel comprises a device or ring at each end to connect it to the rear strap and the two front straps. These straps are connected to a single belt or combined/integrated with briefs.

The belt or the briefs comprise an insert or a material in the back in the form of a rigid narrow and short panel maintaining its flat form.

In brief, the waistband forming a belt, or the briefs are painlessly placed over the gluteal cleft relief at the iliac wings. The waistband or briefs are reinforced in their rear third by an insert preventing the waistband or the waistband of the briefs from giving under tension.

In this way, overall comfortable support the whole ensemble is provided.

### BRIEF DESCRIPTION OF THE DRAWINGS

This invention is described in greater detail below by means of the design methods shown in the attached drawings, in which:

FIG. 1 schematic plan view of the device according to the invention in the form of a cushion equipped with a short and rigid panel maintaining its flat form;

FIG. 2A front view of the entire cushion device installed according to a first embodiment;

FIG. 2B rear view of the entire cushion device installed according to a first embodiment;

FIG. 3 schematic view of the rectangular cushion and belt at the rear attachment point;

FIG. 4A detail view of a rear attachment embodiment;

FIG. 4B detail view of two front attachment points according to one embodiment of the invention;

FIG. 5 perspective view of a first embodiment of the briefs;

FIG. 6A front view of another embodiment of the briefs with an integrated cushion;

FIG. 6B rear view of the briefs in FIG. 6A;

FIG. 6C view of the double front wall;

FIG. 6D view of the front part of the brief with the integrated retaining device as shown in FIG. 6A with the cushion having its front straps passing through the holes and around the coupled rings;

FIG. 7 simplified diagram of the female device according to the state of the art; and

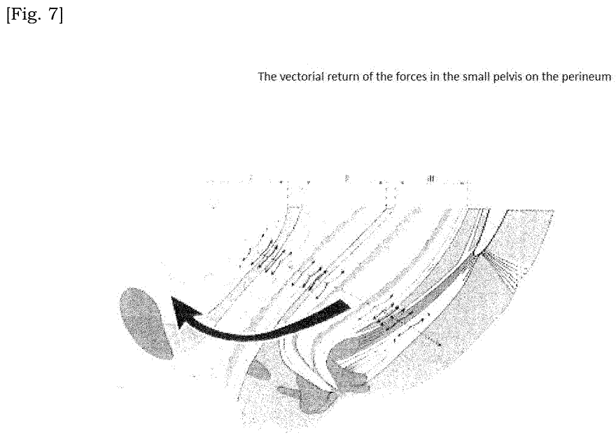

FIG. 8 anatomical diagram showing the complementary transmission of forces by a retaining device according to the invention placed under the back of the perineum.

### DESCRIPTION OF ILLUSTRATIVE EMBODIMENTS

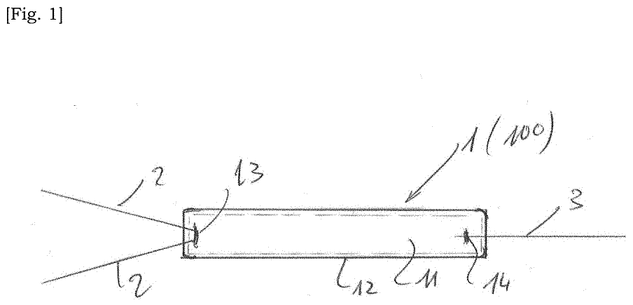

The schematic top view of FIG. 1 shows a cushion 1 according to the invention consisting of a rigid, narrow and short panel 11, maintaining its flat form, housed in a flexible envelope 12. The panel is equipped with a front attachment 13 and a rear attachment 14. The rear attachment 14 is fitted with a rear strap 2 and the front attachment is fitted with two front straps 3.

The rigid, narrow and short panel 11, maintaining its flat form, is about 5 to 11 cm long and about 2 cm wide. The flexible envelope 12 enclosing the rigid panel is a free envelope or a fixed envelope or is integrated to the panel 11.

According to one embodiment, cushion 1 is made by two-part injection of two plastics, a rigid material, maintaining its flat form, forming the insert 11 and a biocompatible flexible material forming the envelope 12.

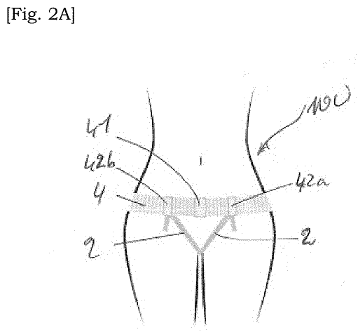

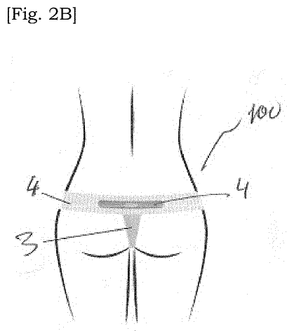

The external directional adjustable support device 100 according to the invention comprises a cushion 1 as shown in FIG. 1 and a belt 4 as shown in FIGS. 2A and 2B.

Belt 4 is a wide, elastic belt with an optional 41 fastener for its adjustment. At the front, the belt 4 has two attachment points 42a, 42b, and at the rear a rear attachment point 43 attached to a rear reinforcement 44.

FIG. 2A shows the two front straps 2, connected to each other at the attachment points 42a, 42b respectively, diverging in a V-shape.

FIG. 2B shows the rear strap 3, connected to the rear attachment point 43. The front straps are preferably elastic and rise towards the waistband, spreading out along the folds of the groin and through the belt 4. They allow tension adjustment at any time while the support device 100 is being worn.

In brief, the waistband 4 is painlessly placed over the gluteal cleft relief at the iliac wings. The waistband formed by the belt 4 is reinforced in its rear third by a reinforcement 44 preventing the belt from sagging when under tension to allow the comfortable wearing of the entire support device 100.

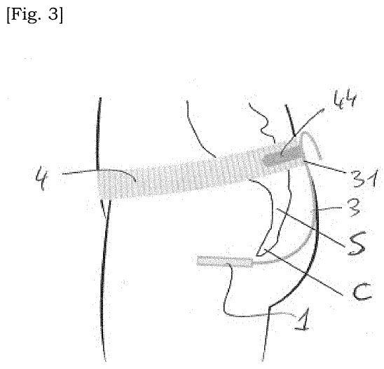

The anatomical diagram in FIG. 3 schematically depicts the positioning of belt 4 with its rear reinforcement 44 and the rear attachment point. Cushion 1 is installed in front of the coccyx C.

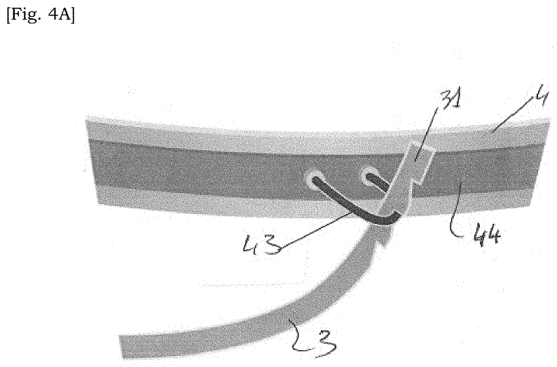

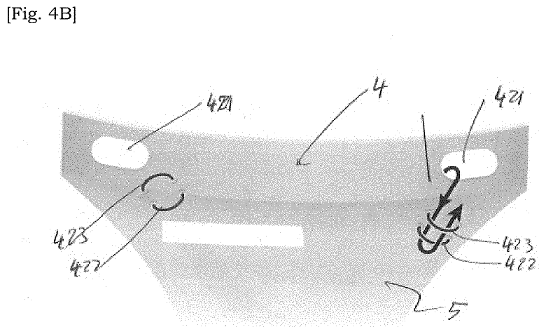

FIG. 4A shows an embodiment of the rear attachment point 43 and the rear strap 3, provided with teeth to form a rack-like arrangement for adjustable attachment in the buckle formed by the rear attachment point 43 connected to the reinforcement 44 of the belt 4. Rear adjustment is made after the belt 4 is put on. The adjusting device consists of the combination of the ring 43, reinforcement 44 and the toothed part at the end of the rear strap 3. This adjustment remains fixed once made. Thus, the rear end of cushion 1 is placed permanently against the coccyx C.

FIG. 4B shows the two front attachment points 42a, 42b of the belt 4 shown here integrated into briefs 5.

The belt has two holes 421 for the passage of the front straps 2 on the right and on the left and, at these holes, two coupled rings 422, 423 allowing the front strap 2 to pass in an S-shape through the first ring 422 and then around the second ring 423 so that the two loops of strap 2 are locked between the two rings 422, 423 by the pull of strap 2 connected to the front end of the cushion 1.

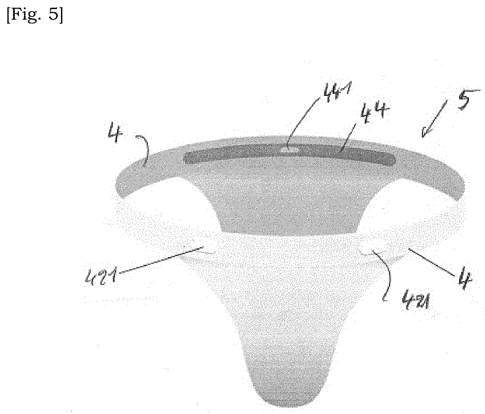

FIG. 5 is a highly schematic perspective view of a belt 4 integrated with briefs 5. Belt 4 has the structure described above with two holes 421 at the front for the passage of the front straps 2 and at the rear a reinforcement 44 with a hole 441 for the passage of the rear strap 3. This opening 441 can be made in the reinforcement 4 or in the rear part of the briefs below the waistband 4.

The rear attachment point 43 on the outside the belt 4 is not visible in FIG. 5.

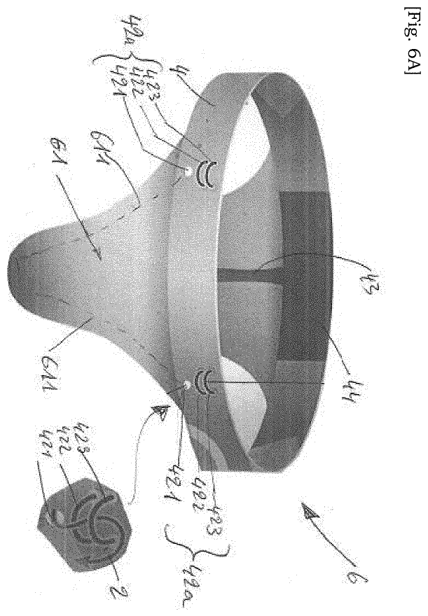

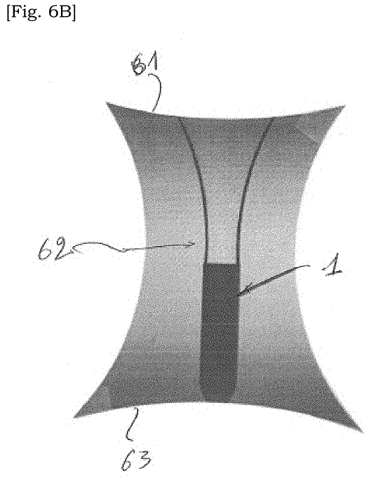

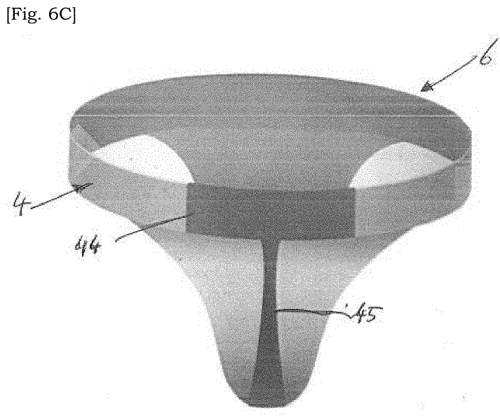

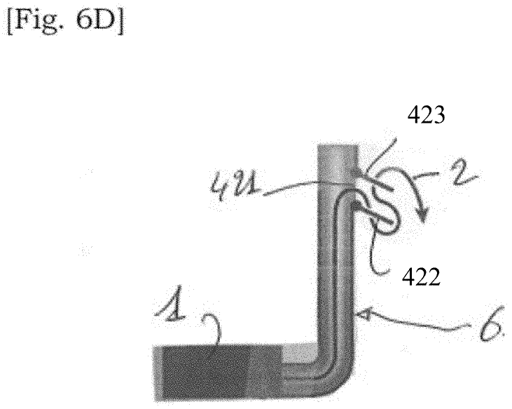

FIGS. 6A, 6B, 6C show another embodiment of briefs 6 integrating cushion 1 into the briefs and thus combining to form the external directional support device 100 according to the invention.

FIG. 6A is a perspective view of the front 61 of briefs 6 with belt 4 corresponding to the embodiments described above.

FIG. 6B shows the integration of cushion 1 in the intermediate part 62 of briefs 6. Cushion 1 supports the two front straps 2. At the back (FIG. 6C) briefs 6 have an integrated reinforcement in the rear part 63 of the briefs, connected to the belt 4 and its reinforcement 44 and extension 45.

The two front straps 2 run through the double front wall of the briefs in two channels 611 opening out under or at waistband 4, near the two rings 42a,b. The front straps 2, not shown in FIG. 6A, extend through the two holes 421 to enclose the two loops 422, 423 of each front attachment point 42a,b. (see also FIG. 6C).

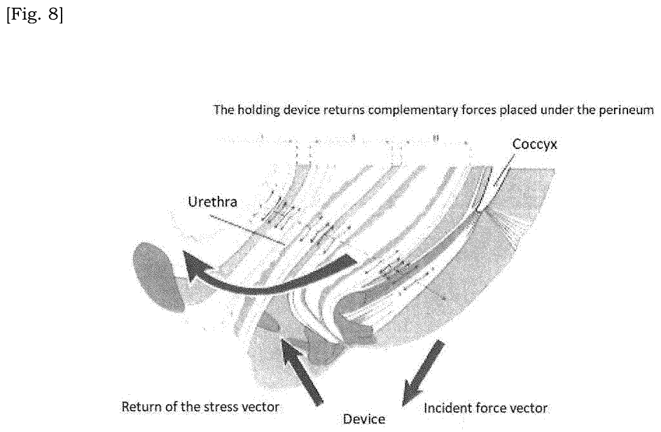

FIG. 8 comprises a curved arrow depicting the vectorial return of the forces in the small pelvis on the perineum.

In greater detail, the abdominal effort causes the organs to slide between each other (red arrow). The organs are elastic and held together by the soft connective tissue (frictional stresses). These movements, retained by the ligaments (pubourethral), cause the plicature of the tissues on one other, like the folding of a garden hose. This plicature stops the flow and is referred to as the functional area of the urethra.

The invention enables redirecting the forces such that this plication can be done, using a narrow, rigid and short panel maintaining its flat, narrow form. This panel is adjustable against the hard part of the small pelvis (the coccyx) at the back and as indicated, the device according to the invention is adjustable thanks to the adjustment of the front tension belt. Depending on the intensity of the abdominal forces, the return of the pressure forces can be redirected more or less upwards towards the pubic bony support. The device blocked at the back by the bony support of the coccyx can thus be adjusted and adapted throughout the day. The adjustable tension of the front straps allows it. In fact, the device is narrow and thus mobile between the other lateral bony reliefs of the branches of the pubis and the ischium.

If the connective tissues loosen, or the pubourethral ligaments are torn, there is nothing to hold the tissue together and the plication becomes non effective. Then the urethra remains straight, allowing the liquid to go through. (urinary incontinence due to urethro-vesical hypermobility).

The invention redirects the stresses so that this plicature can take place.

It consists of a rigid, narrow panel.

This panel is adjustable against the hard part of the small pelvis (the coccyx) at the rear.

Its position on the rear axis of rotation is adjustable to redirect the vectors of the reflected stresses to a variable height.

Its length is no more than the rear two-thirds of the perineum, in order to maintain mobility independent of the anterior third. Its width is no greater than the vaginal cleft between the labia minora, in order to maintain the independent mobility of the lateral parts of the perineum.

This short, narrow, adjustable rigid panel redirects the direction of friction due to the quasi-static transmission of abdominal pressure. A plicature may recur, and the functional area and urethra (also called the sphincter) may become active.

### LIST OF MAIN COMPONENTS

100 External directional support device
1 Cushion
11 Rigid narrow and short panel maintaining its flat form
12 Envelope
13 Front attachment
14 Rear attachment
2 front straps
3 rear strap
31 End of the rear strap
4 Belt
41 Closing catch
42a,b Front attachment point/double ring
421 Hole
422, 423 Coupled rings
43 Rear attachment point, ring
44 Reinforcement
45 Rear reinforcement extension
441 Opening
5 Briefs combined with a retaining device
6 Briefs with integrated retaining device

1. A device for the external adjustable support (100) of the female urethra during effort,
characterized in that it consists in
a rigid rectangular cushion (1) comprising a narrow, rigid and short panel 11 maintaining its flat form, with a back strap (3) and two front straps (2), and
the cushion covers the posterior part of the perineum an abdominal belt (4) with one rear attachment point (43) and two front attachment points (42a,b) which adjustably receive respectively the one rear strap (3) and two front straps (2) to hold the rigid cushion (1) against the rear and lower perineal area, starting from the coccyx.

2. The directional support device (100) according to claim 1,
characterized in that
the belt (4) has a rear reinforcement (4) carrying the rear attachment point (43).

3. The directional support device (100) according to claim 1,
characterized in that
the belt (4) is combined with briefs (6).

4. The directional support device (100) according to claim 3,
characterized in that
the rigid cushion (1) with a narrow, rigid and short panel maintaining its flat form is integrated in the intermediate part (62) of the briefs (6) connecting the front part (61) and the rear part (63).

5. The directional support device (100) according to claim 4,
characterized in that
the briefs (6) have a reinforced rear part (63) at the belt (4) supporting the rear attachment point (43) and a rigid rear part connected to the reinforcement of the gluteal cleft part of the briefs.

6. The directional support device (100) according to claim 4,
characterized in that
the briefs (6) have a front part (61) provided with a double wall forming a passage channel for each of the front straps to join the two adjustable attachment points on the outside.

7. The directional support device according to claim 1,
characterized in that
the front external adjustable attachment points (42a,b) each comprise two coupled rings (422, 423) surmounting the exit hole (421) of the corresponding front strap (2).

8. The directional support device according to claim 1,
characterized in that
the rear adjustable attachment point (43) includes a through buckle attached to the rear reinforcement (44) and the strap has a toothed snap end for hooking the strap (3) to one of the teeth of the buckle to complete the adjustment,
or there is no rear adjustable attachment because of the continuity between posterior gluteal reinforcement, the posterior belt, and the perineal support.

9. The directional support device according to claim 4,
characterized in that
the rigid cushion (1) with a narrow, rigid and short panel maintaining its flat form is integrated in the rear lower region of the intermediate part (62) of the briefs (6), while the front of the cushion (1) has two straps passing through the two routing channels of the double front wall to join the two front attachment points (42a, b).

10. The directional support device according to claim 6,
characterized in that
the front external adjustable attachment points (42a,b) each comprise two coupled rings (422, 423) surmounting the exit hole (421) of the corresponding front strap (2).

11. The directional support device according to claim 6,
characterized in that
the rear adjustable attachment point (43) includes a through buckle attached to the rear reinforcement (44) and the strap has a toothed snap end for hooking the strap (3) to one of the teeth of the buckle to complete the adjustment,
or there is no rear adjustable attachment because of the continuity between posterior gluteal reinforcement, the posterior belt, and the perineal support.

12. The directional support device according to claim 5,
characterized in that
the rigid cushion (1) is integrated in the rear lower region of the intermediate part (62) of the briefs (6), while the front of the cushion (1) has two straps passing through the two routing channels of the double front wall to join the two front attachment points (42a, b).

13. The directional support device according to claim 6,
characterized in that
the rigid cushion (1) is integrated in the rear lower region of the intermediate part (62) of the briefs (6), while the front of the cushion (1) has two straps passing through the two routing channels of the double front wall to join the two front attachment points (42a, b).
